(19) United States
(12) Patent Application Publication
(10) Pub. No.: US 2009/0105196 A1
(43) Pub. Date: Apr. 23, 2009

(54) USE OF CREATINE COMPOUNDS TO TREAT DERMATITIS

(76) Inventors: Belinda Tsao NIVAGGIOLI, Atherton, CA (US); Maisie Wong-Paredes, Sacramento, CA (US)

Correspondence Address: LAHIVE & COCKFIELD, LLP;FLOOR 30, SUITE 3000, ONE POST OFFICE SQUARE, BOSTON, MA, 02109, US

(21) Appl. No.: 12/144,086

(22) Filed: Jun. 23, 2008

Related U.S. Application Data
(60) Provisional application No. 60/936,733, filed on Jun. 22, 2007.

(51) Int. Cl. A61K 31/661; A61K 31/195; A61P 17/00

(52) U.S. Cl. 514/137; 514/565

(57) ABSTRACT

Creatine compounds for the treatment of dermatitis are described.

### RELATED APPLICATIONS

This application claims priority from U.S. Provisional Patent Application No. 60/936,733, filed on Jun. 22, 2007. The contents of the foregoing application are hereby incorporated by reference in their entirety.

### BACKGROUND OF THE INVENTION

The skin is constantly exposed to the elements, making it susceptible to a variety of problems, including dermatitis. Dermatitis is an inflammation of the upper layers of the skin, which can cause itching, blisters, rash, redness, swelling, scabbing, and scaling. There are different types of dermatitis, with some affecting only specific parts of the body. The most common form of dermatitis is contact dermatitis, which results from direct contact with one of many irritants or allergens. The resulting rash is itchy and usually confined to a specific area. While dermatitis is a common condition which is not life threatening, it can make those who suffer from the condition uncomfortable and lead to serious complications. Sometimes, the open sores and fissures that occur with dermatitis can become infected and result in conditions that can be potentially life threatening (e.g., cellulitis).

### SUMMARY OF THE INVENTION

The present invention pertains, at least in part, to methods for treating a subject for dermatitis, by administering to the subject an effective amount of a creatine compound, e.g., compounds of the formula:

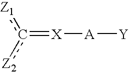

and pharmaceutically acceptable salts thereof.

The present invention also pertains, at least in part, to a method for treating dermatitis in a subject by modulating the energy of skin by administering to the subject an effective amount of a creatine compound.

The present invention also pertains, at least in part, to compositions for treating dermatitis in a subject, in which the compositions comprise an effective amount of a creatine compound and a pharmaceutically acceptable carrier. The invention also includes compositions formulated for topical administration.

The present invention pertains, at least in part, to a packaged pharmaceutical composition and instructions for using the compositions of the invention for the treatment of dermatitis.

### DETAILED DESCRIPTION OF THE INVENTION

### I. Methods

The present invention pertains, at least in part, to a method for treating a subject for dermatitis by administering to the subject an effective amount of a creatine compound.

The term “treated,” “treating” or “treatment” includes therapeutic and/or prophylactic treatment of dermatitis. The treatment includes the diminishment or alleviation of at least one symptom associated with dermatitis. For example, treatment can be diminishment of one or several symptoms of dermatitis (e.g., dry and itchy skin, rash, blisters, scales) or complete eradication of one or more symptoms of dermatitis or the condition (e.g., dermatitis) itself.

The term “subject” includes living organisms capable of suffering from or at risk of dermatitis (e.g., mammals). Examples of subjects at risk may also include those who may be immune compromised, prone to allergies or asthma. Examples of subjects include humans, dogs, cats, horses, cows, goats, rats and mice. The term “subject” also includes include transgenic species. In one embodiment, the subject is a human.

The term “dermatitis” includes a condition characterized by the inflammation of the upper layers of the skin, causing itching, blisters, redness, swelling, oozing, scabbing and scaling. Examples of types of dermatitis includes, but is not limited to, atopic dermatitis, contact dermatitis, generalized exfoliative dermatitis, neurodermatitis, nummular dermatitis, seborrheic dermatitis, stasis dermatitis, perioral dermatitis or pompholyx.

In a further embodiment, the creatine compound is administered in combination with a pharmaceutically acceptable carrier. Examples of such carriers include those suitable for topical or oral administration.

The term “administered,” “administering” or “administration” includes routes of administration which allow the creatine compounds to perform their intended function(s) of preventing, ameliorating, arresting, and/or eliminating dermatitis in a subject. Examples of routes of administration which may be used include injection, topical, oral, subcutaneous, intravenous, parenterally, intraperitoneally, inhalation and transdermal. Depending on the route of administration, the creatine compound may be coated with or in a material to protect it from the natural conditions which may detrimentally affect its ability to perform its intended function. The administration of the compound is done at dosages and for periods of time effective to reduce, ameliorate or eliminate the symptoms of dermatitis. Dosage regimes may be adjusted for purposes of improving the therapeutic or prophylactic response of the compound. For example, several divided doses may be administered daily or the dose may be proportionally reduced as indicated by the exigencies of the therapeutic situation. Suitable pharmaceutical vehicles or dosage forms for injectable compositions, implants, and systemic administration are known. The creatine compounds may be administered topically to the skin and can be formulated into a variety of topically administrable compositions, such as lotions, solutions, suspensions, gels or ointments.

In one embodiment, the method for treating dermatitis in a subject by administering an effective amount of a creatine compound, optionally in combination with an anti-inflammatory agent, does not comprise administering an alpha-ketoacid or an alpha-hydroxy acid to the subject.

The term “therapeutically effective amount” or “effective amount” includes an amount of the creatine compound that is sufficient in treating or preventing dermatitis. A therapeutically effective amount can be determined on an individual basis and will be based, in part, on the severity of the symptoms and the activity of the specific creatine compound. Thus, a therapeutically effective amount of a creatine compound can be determined by one of ordinary skill in the art using no more than routine experimentation in clinical management.

In another embodiment, the method further comprises administering the creatine compound in combination with an anti-inflammatory agent. Examples of anti-inflammatory agents include, but are not limited to, steroidal anti-inflammatory agents and non-steroidal anti-inflammatory agents (NSAIDs).

A safe and effective amount of an anti-inflammatory agent may be added to the methods and compositions of the present invention, preferably from about 0.1% to about 10%, more preferably from about 0.5% to about 5%, of the composition. The exact amount of anti-inflammatory agent to be used in the methods and compositions will depend on the particular anti-inflammatory agent utilized since such agents vary widely in potency.

Steroidal anti-inflammatory agents, including but not limited to, corticosteroids such as hydrocortisone, hydroxyltriamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used.

A second class of anti-inflammatory agents which is useful in the methods and compositions of the present invention includes the nonsteroidal anti-inflammatory agents (NSAIDs). Suitable NSAIDs include, but are not limited to salicylates (e.g., acetylsalicylic acid (aspirin), amoxiprin, benorylate/benorilate, choline magnesium salicylate, diflunisal, ethenzamide, faislamine, methyl salicylate, magnesium salicylate, salicyl salicylate, salicylamide), arylalkanoic acids (e.g., diclofenac, aceclofenac, acemetacin, alclofenac, bromfenac, etodolac, indometacin, nabumetone, oxametacin, proglumetacin, sulindac, tolmetin), 2-arylpropionic acids (profens) (e.g., ibuprofen, alminoprofen, benoxaprofen, carprofen, dexibuprofen, dexketoprofen, fenbufen, fenoprofen, flunoxaprofen, flurbiprofen, ibuproxam, indoprofen, ketoprofen, ketorolac, loxoprofen, naproxen, oxaprozin, pirprofen, suprofen, tiaprofenic acid), N-arylanthranilic acids (fenamic acids) (e.g., mefenamic acid, flufenamic acid, meclofenamic acid, tolfenamic acid), pyrazolidine derivatives (e.g., phenylbutazone, ampyrone, azapropazone, clofezone, kebuzone, metamizole, mofebutazone, oxyphenbutazone, phenazone, phenylbutazone, sulfinpyrazone), oxicams (e.g., piroxicam, droxicam, lomoxicam, meloxicam, tenoxicam), COX-2 inhibitors (e.g., celecoxib, etoricoxib, lumiracoxib, parecoxib, rofecoxib, valdecoxib) and sulphonanilides (e.g., nimesulide).

The variety of compounds encompassed by this group are well-known to those skilled in the art. For detailed disclosure of the chemical structure, synthesis, side effects, etc., of non-steroidal anti-inflammatory agents, reference may be had to standard texts, including Antiinflammatory and Anti-Rheumatic Drugs, K. D. Rainsford, Vol. I-III, CRC Press, Boca Raton, (1985), and Anti-inflammatory Agents. Chemistry and Pharmacology, 1, R. A. Scherrer, et al., Academic Press, New York (1974). Mixtures of these non-steroidal anti-inflammatory agents may also be employed, as well as the pharmaceutically-acceptable salts and esters of these agents. For example, etofenamate, a flufenamic acid derivative, is particularly useful for topical application. Yet another class of anti-inflammatory agents which are useful in the present invention are those disclosed in U.S. Pat. No. 4,912,248, Maeller, issued Mar. 27, 1990. This patent discloses compounds and diastereomeric mixtures of specific 2-naphthyl-containing ester compounds, especially naproxen ester and naproxol ester compounds, having two or more chiral centers. Finally, so-called “natural” anti-inflammatory agents are useful in the present invention. For example, candelilla wax, alpha bisabolol, aloe vera, Manjistha (extracted from plants in the genus Rubia, particularly Rubia Cordifolia), and Guggal (extracted from plants in the genus Commiphora, particularly Commiphora Mukul), may be used.

The language “in combination with” an anti-inflammatory agent includes co-administration of the creatine compound with the anti-inflammatory agent; administration of the creatine compound first, followed by the anti-inflammatory agent and administration of the anti-inflammatory agent first, followed by the creatine compound.

The invention also pertains, at least in part, to a method for treating dermatitis in a subject by modulation of energy of skin of the subject by administering an effective amount of a creatine compound. The modulation of energy of skin may occur by modulating creatine kinase activity.

The term “modulation” includes the up regulation or down regulation of pathways or systems such as, but not limited to, the creatine kinase/creatine phosphate energy system. Examples of modulation include, but are not limited to, for example, increases in the level of phosphorylation by creatine kinase. For example, the level of phosphorylation may be increased by at least about 5% or greater, by at least about 10% or greater, by at least about 15% or greater, by at least about 20% or greater, by at least about 30% or greater, by at least about 40% or greater, by at least about 50% or greater or by at least about 75% or greater.

The term “energy of skin” includes any form of energy in the skin. Examples of energy in the skin include the energy generated by kinases though phosphorylation, e.g., the creatine kinase/creatine phosphate energy system.

### II. Creatine Compounds

The term “creatine compounds” includes compounds which modulate one or more of the structural or functional components of the creatine kinase/phosphocreatine system. The term includes creatine, creatine monohydrate, creatine phosphate and analogs thereof, compounds which mimic their activity, and salts of these compounds.

The term “creatine compounds” also includes compounds of the general formula I:

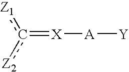

and pharmaceutically acceptable salts thereof, where:
a) Y is selected from the group consisting of: —CO₂H, —NHOH, —NO₂, —SO₃H, —C(═O)NHSO₂J and —P(═O)(OH)(OJ), wherein J is selected from the group consisting of: hydrogen, C₁-C₆ straight chain alkyl, C₃-C₆ branched alkyl, C₂-C₆ alkenyl, C₃-C₆ branched alkenyl, and aryl;
b) A is selected from the group consisting of: C, CH, C₁-C₅ alkyl, C₂-C₅ alkenyl, C₂-C₅ alkynyl, and C₁-C₅ alkoyl chain, each having 0-2 substituents which are selected independently from the group consisting of:
   1) K, where K is selected from the group consisting of: C₁-C₆ straight alkyl, C₂-C₆ straight alkenyl, C₁-C₆ straight alkoyl, C₃-C₆ branched alkyl, C₃-C₆ branched alkenyl, and C₄-C₆ branched alkoyl, K having 0-2 substituents independently selected from the group consisting of: bromo, chloro, epoxy and acetoxy;
   2) an aryl group selected from the group consisting of: a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy; and
   3) —NH-M, wherein M is selected from the group consisting of: hydrogen, C₁-C₄ alkyl, C₂-C₄ alkenyl, C₁-C₄ alkoyl, C₃-C₄ branched alkyl, C₃-C₄ branched alkenyl, and C₄ branched alkoyl;
c) X is selected from the group consisting of NR₁, CHR₁, CR₁, O and S, wherein R₁ is selected from the group consisting of:
   1) hydrogen;
   2) K where K is selected from the group consisting of: C₁-C₆ straight alkyl, C₂-C₆ straight alkenyl, C₁-C₆ straight alkoyl, C₃-C₆ branched alkyl, C₃-C₆ branched alkenyl, and C₄-C₆ branched alkoyl, K having 0-2 substituents independently selected from the group consisting of: bromo, chloro, epoxy and acetoxy;
   3) an aryl group selected from the group consisting of a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy;
   4) a C₅-C₉ α-amino-ω-methyl-ω-adenosylcarboxylic acid attached via the ω-methyl carbon;
   5) a C₅-C₉ α-amino-ω-aza-ω-methyl-w-adenosylcarboxylic acid attached via the ω-methyl carbon; and
   6) a C₅-C₉ α-amino-ω-thia-ω-methyl-w-adenosylcarboxylic acid attached via the w-methyl carbon;
d) Z₁ and Z₂ are chosen independently from the group consisting of: ═O, —NHR₂, —CH₂R₂, —NR₂OH; wherein Z₁ and Z₂ may not both be ═O and wherein R₂ is selected from the group consisting of:
   1) hydrogen;
   2) K, where K is selected from the group consisting of: C₁-C₆ straight alkyl; C₂-C₆ straight alkenyl, C₁-C₆ straight alkoyl, C₃-C₆ branched alkyl, C₃-C₆ branched alkenyl, and C₄-C₆ branched alkoyl, K having O-2 substituents independently selected from the group consisting of: bromo, chloro, epoxy and acetoxy;
   3) an aryl group selected from the group consisting of a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy;
   4) a C₄-C₈ α-amino-carboxylic acid attached via the ω-carbon;
   5) B, wherein B is selected from the group consisting of: —CO₂H, —NHOH, —SO₃H, —NO₂, OP(═O)(OH)(OJ) and —P(═O)(OH)(OJ), wherein J is selected from the group consisting of: hydrogen, C₁-C₆ straight alkyl, C₃-C₆ branched alkyl, C₂-C₆ alkenyl, C₃-C₆ branched alkenyl, and aryl, wherein B is optionally connected to the nitrogen via a linker selected from the group consisting of: C₁-C₂ alkyl, C₂ alkenyl, and C₁-C₂ alkoyl;
   6) -D-E, wherein D is selected from the group consisting of: C₁-C₃ straight alkyl, C₃ branched alkyl, C₂-C₃ straight alkenyl, C₃ branched alkenyl, C₁-C₃ straight alkoyl, aryl and aroyl; and E is selected from the group consisting of: —(PO₃)ₙ NMP, where n is 0-2 and NMP is ribonucleotide monophosphate connected via the 5′-phosphate, 3′-phosphate or the aromatic ring of the base; —[P(═O)(OCH₃)(O)]ₘ-Q, where m is 0-3 and Q is a ribonucleoside connected via the ribose or the aromatic ring of the base; —[P(═O)(OH)(CH₂)]ₘ-Q, where m is 0-3 and Q is a ribonucleoside connected via the ribose or the aromatic ring of the base; and an aryl group containing 0-3 substituents chosen independently from the group consisting of: Cl, Br, epoxy, acetoxy, —OG, —C(═O)G, and —CO₂G, where G is independently selected from the group consisting of: C₁-C₆ straight alkyl, C₂-C₆ straight alkenyl, C₁-C₆ straight alkoyl, C₃-C₆ branched alkyl, C₃-C₆ branched alkenyl, C₄-C₆ branched alkoyl, wherein E may be attached to any point to D, and if D is alkyl or alkenyl, D may be connected at either or both ends by an amide linkage; and
   7) -E, wherein E is selected from the group consisting of —(PO₃)ₙNMP, where n is 0-2 and NMP is a ribonucleotide monophosphate connected via the 5′-phosphate, 3′-phosphate or the aromatic ring of the base; —[P(═O)(OCH₃)(O)]ₘ-Q, where m is 0-3 and Q is a ribonucleoside connected via the ribose or the aromatic ring of the base; —[P(═O)(OH)(CH₂)]ₘ-Q, where m is 0-3 and Q is a ribonucleoside connected via the ribose or the aromatic ring of the base; and an aryl group containing 0-3 substituents chose independently from the group consisting of: Cl, Br, epoxy, acetoxy, —OG, —C(═O)G, and —CO₂G, where G is independently selected from the group consisting of: C₁-C₆ straight alkyl, C₂-C₆ straight alkenyl, C₁-C₆ straight alkoyl, C₃-C₆ branched alkyl, C₃-C₆ branched alkenyl, C₄-C₆ branched alkoyl; and if E is aryl, E may be connected by an amide linkage;
e) if R₁ and at least one R₂ group are present, R₁ may be connected by a single or double bond to an R₂ group to form a cycle of 5 to 7 members;
f) if two R₂ groups are present, they may be connected by a single or a double bond to form a cycle of 4 to 7 members; and
g) if R₁ is present and Z₁ or Z₂ is selected from the group consisting of —NHR₂, —CH₂R₂ and —NR₂OH, then R₁ may be connected by a single or double bond to the carbon or nitrogen of either Z₁ or Z₂ to form a cycle of 4 to 7 members.

Creatine (also known as N-(aminoiminomethyl)-N-methylglycine; methylglycosamine or N-methyl-guanido acetic acid) is a well-known substance. (See, The Merck Index, Eleventh Edition, No. 2570 (1989).

Creatine is phosphorylated chemically or enzymatically by creatine kinase to generate creatine phosphate, which also is well-known (see, The Merck Index, No. 7315). Both creatine and creatine phosphate (phosphocreatine) can be extracted from animal tissue or synthesized chemically. Both are commercially available.

Cyclocreatine is an essentially planar cyclic analog of creatine. Although cyclocreatine is structurally similar to creatine, the two compounds are distinguishable both kinetically and thermodynamically. Cyclocreatine is phosphorylated efficiently by creatine kinase in the forward reaction both in vitro and in vivo. Rowley, G. L., J. Am. Chem. Soc. 93: 5542-5551 (1971); McLaughlin, A. C. et. al., J. Biol. Chem. 247, 4382-4388 (1972).

The phosphorylated compound phosphocyclocreatine is structurally similar to phosphocreatine; however, the phosphorous-nitrogen (P—N) bond of cyclocreatine phosphate is more stable than that of phosphocreatine. LoPresti, P. and M. Cohn, Biochem. Biophys. Acta 998: 317-320 (1989); Annesley, T. M. and J. B. Walker, J. Biol. Chem. 253; 8120-8125, (1978); Annesley, T. M. and J. B. Walker, Biochem. Biophys. Res. Commun. 74:185-190 (1977).

3-Guanidinopropionic acid (3-GPA) is an endogenous metabolite found in animals and humans (Hiraga et. al., J. of Chromatography vol 342, 269-275, 1985; Watanabe et. al., Guanidines edited by Mori et. al., Plenum, N.Y., 49-58, 1983). The compound is available from Sigma chemicals and is an extensively studied analog of creatine.

Guanidino acetate is yet another analog of creatine and is a precursor of creatine in its biosynthetic pathway.

Guanidino benzoic acids are structurally related to creatine. Also compounds that attach amino acid like molecules covalently to creatine are creatine compounds of interest. Examples are creatine-ascorbate and creatine-pyruvate. Other types of molecules could be covalently attached.

Creatine analogs and other agents which act to interfere with the activity of creatine biosynthetic enzymes or with the creatine transporter are useful in the present method of treating or preventing dermatitis. Thus the effects of such compounds can be direct or indirect, operating by mechanisms including, but not limited to, influencing the uptake or biosynthesis of creatine, the function of the creatine phosphate shuttle, enzyme activity, or the activity of associated enzymes, or altering the levels of substrates or products of a reaction to alter the velocity of the reaction.

Creatine, creatine phosphate and many creatine analogs are commercially available. Additionally, analogs of creatine may be synthesized using conventional techniques. For example, creatine can be used as the starting material for synthesizing at least some of the analogs encompassed by formula I. Appropriate synthesis reagents, e.g. alkylating, alkenylating or alkynylating agents may be used to attach the respective groups to target sites. Alternatively, reagents capable of inserting spacer groups may be used to alter the creatine structure. Sites other than the target site are protected using conventional protecting groups while the desired sites are being targeted by synthetic reagents.

If the creatine analog contains a ring structure, then the analog may be synthesized in a manner analogous to that described for cyclocreatine (Wang, T., J. Org, Chem, 39:3591-3594 (1974)). The various other substituent groups may be introduced before or after the ring is formed.

Many creatine analogs have been previously synthesized and described (Rowley et al., J. Am. Chem. Soc. 93:5542-5551 (1971); McLaughlin et al., J. Biol. Chem. 247:4382-4388 (1972); Nguyen, A. C. K., “Synthesis and enzyme studies using creatine analogs”, Thesis, Dept. of Pharmaceutical Chemistry, Univ. Calif., San Francisco (1983); Lowe et al., J. Biol. Chem. 225:3944-3951 (1980); Roberts et al., J. Biol. Chem. 260:13502-13508 (1985); Roberts et al., Arch. Biochem. Biophys. 220:563-571 (1983), and Griffiths et al., J. Biol. Chem. 251:2049-2054 (1976)). The contents of all of the aforementioned references are expressly incorporated by reference. Further to the aforementioned references, Kaddurah-Daouk et al. (WO92/08456; WO90/09192; U.S. Pat. No. 5,324,731; U.S. Pat. No. 5,321,030) also provide citations for the synthesis of a plurality of creatine analogs. Also the synthesis of creatine-pyruvate and creatine ascorbate has been described in a series of patents WPI 98-481123/199841; WPI 98-457997/199840; WPI 98-387651/199833). The contents of all the aforementioned references and patents are incorporated herein by reference.

Creatine compounds which currently are available or have been synthesized include, for example, creatine, β-guanidinopropionic acid, creatine monohydrate, guanidinoacetic acid, creatine phosphate disodium salt, cyclocreatine, homocyclocreatine, phosphinic creatine, homocreatine, ethylcreatine, cyclocreatine phosphate dilithium salt, guanidinoacetic acid phosphate disodium salt, 4 guanidino benzoic acid and derivatives, creatine pyruvate, creatine ascorbate, creatine citrate among others.

Creatine phosphate compounds also can be synthesized chemically or enzymatically. The chemical synthesis is well known. Annesley, T. M. Walker, J. B., Biochem. Biophys. Res. Commun., (1977), 74, 185-190; Cramer, F., Scheiffele, E., Vollmar, A., Chem. Ber., (1962), 95, 1670-1682.

Salts of the products may be exchanged to other salts using standard protocols. The enzymatic synthesis utilizes the creatine kinase enzyme, which is commercially available, to phosphorylate the creatine compounds. ATP is required by creatine kinase for phosphorylation, hence it needs to be continuously replenished to drive the reaction forward. It is necessary to couple the creatine kinase reaction to another reaction that generates ATP to drive it forward. The purity of the resulting compounds can be confirmed using known analytical techniques including ¹H NMR, ¹³CNMR Spectra, Thin layer chromatography, HPLC and elemental analysis.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In certain embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Moreover, the term alkyl includes both “unsubstituted alkyls” and “substituted alkyls,” the latter of which refers to alkyl moieties having substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl (benzyl)). The term “alkyl” also includes the side chains of natural and unnatural amino acids.

The term “alkoyl” denotes an alkyl group as defined above, connected through a carbonyl group to the parent molecular residue. Examples include, but are not limited to formyl, acetyl, propionyl, butyryl, iso-butyryl, pivaloyl, and the like.

The term “aryl” includes groups, including 5- and 6-membered single-ring aromatic groups that may include from zero to four heteroatoms, for example, benzene, phenyl, pyrrole, furan, thiophene, thiazole, isothiaozole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles,” “heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “alkenyl” includes unsaturated aliphatic groups analogous in length and possible substitution to the alkyls described above, but that contain at least one double bond.

For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups which include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls,” the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups which include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term alkynyl includes both “unsubstituted alkynyls” and “substituted alkynyls,” the latter of which refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” includes an alkyl group, as defined above, but having from one to five carbon atoms in its backbone structure. “Lower alkenyl” and “lower alkynyl” have chain lengths of, for example, 2-5 carbon atoms.

The term “acyl” includes compounds and moieties which contain the acyl radical (CH₃CO—) or a carbonyl group. It includes substituted acyl moieties. The term “substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes moieties wherein an acyl moiety is bonded to an amino group. For example, the term includes alkylcarbonylamino, arylcarbonylamino, carbamoyl and ureido groups.

The term “aroyl” includes compounds and moieties with an aryl or heteroaromatic moiety bound to a carbonyl group. Examples of aroyl groups include phenylcarboxy, naphthyl carboxy, etc.

The terms “alkoxyalkyl,” “alkylaminoalkyl” and “thioalkoxyalkyl” include alkyl groups, as described above, which further include oxygen, nitrogen or sulfur atoms replacing one or more carbons of the hydrocarbon backbone, e.g., oxygen, nitrogen or sulfur atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulflhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “amine” or “amino” includes compounds where a nitrogen atom is covalently bonded to at least one carbon or heteroatom. The term includes “alkyl amino” which comprises groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

The term “amide,” “amido” or “aminocarbonyl” includes compounds or moieties which contain a nitrogen atom which is bound to the carbon of a carbonyl or a thiocarbonyl group. The term includes “alkaminocarbonyl” or “alkylaminocarbonyl” groups which include alkyl, alkenyl, aryl or alkynyl groups bound to an amino group bound to a carbonyl group. It includes arylaminocarbonyl and arylcarbonylamino groups which include aryl or heteroaryl moieties bound to an amino group which is bound to the carbon of a carbonyl or thiocarbonyl group. The terms “alkylaminocarbonyl,” “alkenylaminocarbonyl,” “alkynylaminocarbonyl,” “arylaminocarbonyl,” “alkylcarbonylamino,” “alkenylcarbonylamino,” “alkynylcarbonylamino,” and “arylcarbonylamino” are included in term “amide.” Amides also include urea groups (aminocarbonylamino) and carbamates (oxycarbonylamino).

The term “carbonyl” or “carboxy” includes compounds and moieties which contain a carbon connected with a double bond to an oxygen atom. The carbonyl can be further substituted with any moiety which allows the compounds of the invention to perform its intended function. For example, carbonyl moieties may be substituted with alkyls, alkenyls, alkynyls, aryls, alkoxy, aminos, etc. Examples of moieties which contain a carbonyl include aldehydes, ketones, carboxylic acids, amides, esters, anhydrides, etc.

The term “ether” includes compounds or moieties which contain an oxygen bonded to two different carbon atoms or heteroatoms. For example, the term includes “alkoxyalkyl” which refers to an alkyl, alkenyl, or alkynyl group covalently bonded to an oxygen atom which is covalently bonded to another alkyl group.

The term “ester” includes compounds and moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

The terms “polycyclyl” or “polycyclic radical” refer to two or more cyclic rings (e.g., cycloalkyls, cycloalkenyls, cycloalkynyls, aryls and/or heterocyclyls) in which two or more carbons are common to two adjoining rings, e.g., the rings are “fused rings.” Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, arylalkylaminocarbonyl, alkenylaminocarbonyl, alkylcarbonyl, arylcarbonyl, arylalkyl carbonyl, alkenylcarbonyl, aminocarbonyl, alkylthiocarbonyl, alkoxyl, phosphate, phosphonato, phosphinato, cyano, amido, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur and phosphorus.

### III. Pharmaceutical Compositions

The present invention pertains, at least in part, to compositions for treating dermatitis in a subject comprising an effective amount of a creatine compound or salt thereof and a pharmaceutically acceptable carrier. The compositions of the present invention may be suitable for topical or oral administration and may further comprise an anti-inflammatory agent.

Packaged pharmaceutical compositions to treat dermatitis are also the subject of the present invention. The packaged compositions include a container holding the creatine compound of the invention in combination with a pharmaceutically acceptable carrier, along with instructions (e.g., instructions for once a day administration, twice a day administration, etc.) for use for the treatment of dermatitis. The package may further comprise an anti-inflammatory agent.

The phrase “pharmaceutically acceptable carrier” includes a pharmaceutically acceptable material, composition or vehicle, such as a liquid or solid filler, diluent, excipient, solvent or encapsulating material, involved in carrying or transporting the creatine compound within or to the subject such that it can performs its intended function, e.g. to treat dermatitis. Each carrier must be “acceptable” in the sense of being compatible with the other ingredients of the formulation and not injurious to the subject. Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds of the invention.

In one embodiment, the pharmaceutical composition comprising a creatine compound and, optionally an anti-inflammatory agent, and a pharmaceutically acceptable carrier has a pH of greater than 4.0. In another embodiment, pharmaceutical composition comprising a creatine compound and, optionally an anti-inflammatory agent, and a pharmaceutically acceptable carrier, does not comprise an alpha-ketoacid and/or an alpha-hydroxy acid, including, but not limited to, 2-hydroxyethanoic acid (e.g., glycolic acid or hydroxyacetic acid), 2-hydroxypropanoic acid (e.g., lactic acid); 2-methyl 2-hydroxypropanoic acid (e.g., methyllacetic acid), 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid (e.g., alpha hydroxylauric acid), 2-hydroxytetradecanoic acid (e.g., alpha hydroxymyristic acid), 2-hydroxyhexadecanoic acid (e.g., alpha hydroxypalmitic acid) 2-hydroxyoctadecanoic acid (e.g., alpha hydroxystearic acid), 2-hydroxyeicosanoic acid (e.g., alpha hydroxyarachidonic acid), 2-phenyl 2-hydroxyethanoic acid (e.g., mandelic acid), 2,2-diphenyl 2-hydroxyethanoic acid (e.g., benzilic acid), 3-phenyl 2-hydroxypropanoic acid (e.g., phenyllacetic acid), 2-phenyl 2-methyl 2-hydroxyethanoic acid (e.g., atrolactic acid), 2-(4′-hydroxyphenyl) 2-hydroxyethanoic acid (e.g., 4-hydroxymandelic acid), 2-(4′-chlorophenyl) 2-hydroxyethanoic acid (e.g., 4-chloromandelic acid), 2-(3′-hydroxy-4′-methoxyphenyl) 2-hydroxyethanoic acid (e.g., 3-hydroxy-4-methoxymandelic acid), 2-(4′-hydroxy-3′-methoxyphenyl) 2-hydroxyethanoic acid (e.g., 4-hydroxy-3-methoxymandelic acid), 3-(2′-hydroxyphenyl) 2-hydroxypropanoic acid (e.g., 3-(2′-hydroxyphenyl)lactic acid), 3-(4′-hydroxyphenyl) 2-hydroxypropanoic acid (e.g., 3-(4′-hydroxyphenyl)lactic acid), 2-(3′,4′-dihydroxyphenyl) 2-hydroxyethanoic acid (e.g., 3,4-dihydroxymandelic acid), 2,3-dihydroxypropanoic acid (e.g., glyceric acid), 2,3,4-trihydroxybutanoic acid and isomers thereof (e.g., erythronic acid, threonic acid), 2,3,4,5-tetrahydroxypentanoic acid and isomers thereof (e.g., ribonic acid, arabinoic acid, xylonic acid, lyxonic acid), 2,3,4,5,6-pentahydroxyhexanoic acid and isomers thereof (e.g., allonic acid, altronic acid, gluconic acid, mannoic acid, gulonic acid, idonic acid, galactonic acid, talonic acid), 2,3,4,5,6,7-hexahydroxyheptanoic acid and isomers thereof (e.g., glucoheptonic acid, galactoheptonic acid, etc.), 2-hydroxypropane-1,3-dioic acid (e.g., tartronic acid), 2-hydroxybutane-1,4-dioic acid (e.g., malic acid), 2,3-dihydroxybutane-1,4-dioic acid (e.g., tartaric acid), 2-hydroxy-2-carboxypentane-1,5-dioic acid (e.g., citric acid), 2,3,4,5-tetrahydroxyhexane-1,6-dioic acid and isomers thereof (e.g., saccharic acid, mucic acid, etc.), gluconolactone, galactonolactone, glucuronolactone, galacturonolactone, gulonolactone, ribonolactone, saccharic acid lactone, pantoyllactone, glucoheptonolactone, mannonolactone, galactoheptonolactone, 2-ketoethanoic acid (e.g., glyoxylic acid), methyl 2-ketoethanoate, 2-Ketopropanoic acid (e.g., pyruvic acid), methyl 2-ketopropanoate (e.g., methyl pyruvate), ethyl 2-ketopropanoate (e.g., ethyl pyruvate), propyl 2-ketopropanoate (e.g., propyl pyruvate), 2-phenyl-2-ketoethanoic acid (e.g., benzoylformic acid), methyl 2-phenyl-2-ketoethanoate (e.g., methyl benzoylformate), ethyl 2-phenyl-2-ketoethanoate (e.g., ethyl benzoylformate), 3-phenyl-2-ketopropanoic acid (e.g., phenylpyruvic acid), methyl 3-phenyl-2-ketopropanoate (e.g., methyl phenylpyruvate), ethyl 3-phenyl-2-ketopropanoate (e.g., ethyl phenylpyruvate), 2-ketobutanoic acid, 2-ketopentanoic acid, 2-ketohexanoic acid, 2-ketoheptanoic acid, 2-ketooctanoic acid, 2-ketododecanoic acid, methyl 2-ketooctanoate, ascorbic acid, quinic acid, isocitric acid, tropic acid, trethocanic acid, 3-chlorolactic acid, cerebronic acid, citramalic acid, agaricic acid, 2-hydroxynervonic acid, aleuritic acid, pantoic acid, glycolyl glycollate (e.g., glycolic acid glycollate, lactyl lactate (e.g., lactic acid lactate), mandelyl mandellate, atrolactyl atrolactate, phenyllactyl phenyllactate, benzilyl benzillate, glycolyl lactate, lactyl glycollate, glycolyl glycolyl glycollate, lactyl lactyl lactate, lactyl glycolyl lactate, glycolyl glycolyl glycolyl glycollate, lactyl lactyl lactyl lactate, glycolyl lactyl glycolyl lactyl glycollate, polyglycolic acid, polylactic acid, glycolide, lactide, mandelide, atrolactide, phenyllactide, benzilide, methyllactide, lactoglycolide and glycolactide.

The term “topical administration” includes methods of delivery such as laying on or spreading on the skin. It involves any form of administration which involves the skin. Examples of compositions suitable for topical administration, include but are not limited to, ointments, lotions, creams, cosmetic formulations, and skin cleansing formulations. Additional examples include aerosols, solids (such as bar soaps) and gels.

The topical pharmaceutical compositions of the present invention may be made into a wide variety of product types. These include, but are not limited to solutions, lotions, creams, beach products, gels, sticks, sprays, pads, ointments, pastes, mousses and cosmetics. These product types may comprise several types of carrier systems including, but not limited to solutions, emulsions, gels and solids.

The topical pharmaceutical compositions of the present invention formulated as solutions typically include a pharmaceutically-acceptable aqueous or organic solvent. The terms “pharmaceutically-acceptable aqueous solvent” and “pharmaceutically-acceptable organic solvent” refer to a solvent which is capable of having dispersed or dissolved therein the active compound, and possesses acceptable safety properties (e.g., irritation and sensitization characteristics). Water is a typical aqueous solvent. Examples of suitable organic solvents include: propylene glycol, butylene glycol, polyethylene glycol (200-600), polypropylene glycol (425-2025), glycerol, 1,2,4-butanetriol, sorbitol esters, 1,2,-6-hexanetriol, ethanol, isopropanol, butanediol, and mixtures thereof. Preferably, these solutions contain from about 0.01% to about 50% of the creatine compound, more preferably from about 0.1% to about 20%, and, for example, between about 0.1% and 10% of the creatine compound; and from about 1% to about 80% of an acceptable aqueous or organic solvent, more preferably from about 1% to about 40%.

If the topical pharmaceutical compositions of the present invention are formulated as an aerosol and applied to the skin as a spray-on, a propellant is added to a solution composition. A more complete disclosure of propellants useful herein can be found in Sagarin, Cosmetics Science and Technology, 2nd Edition, Vol. 2, pp. 443-465 (1972).

Topical pharmaceutical compositions of the present invention may be formulated as a solution comprising an emollient. An example of a composition formulated in this way would be a sunscreen-containing product. Preferably, such compositions contain from about 0.1% to about 50% of the active compound and from about 2% to about 50% of a topical pharmaceutically-acceptable emollient.

As used herein, “emollients” refer to materials used for the prevention or relief of dryness, as well as for the protection of the skin. A wide variety of suitable emollients are known and may be used herein. Sagarin, Cosmetics, Science and Technology, 2nd Edition, Vol. 1, pp. 32-43 (1972), incorporated herein by reference, contains numerous examples of suitable materials.

A lotion can be made from a solution carrier system. Lotions preferably comprise from about 0.1% to about 20%, more preferably from about 1% to about 5%, of the active compound; from about 1% to about 20%, preferably from about 5% to about 10%, of an emollient; and from about 50% to about 90%, preferably from about 60% to about 80%, water.

Another type of product that may be formulated from a solution carrier system is a cream. A cream of the present invention would preferably comprise from about 0.1% to about 20%, more preferably from about 1% to about 5%, of the active compound; from about 5% to about 50%, preferably from about 10% to about 20%, of an emollient, and from about 45% to about 85%, preferably from about 50% to about 75%, water.

Yet another type of product that may be formulated from a solution carrier system is an ointment. An ointment may comprise a simple base of animal or vegetable oils or semi-solid hydrocarbons (oleaginous). Ointments may also comprise absorption ointment bases which absorb water to form emulsions. Ointment carriers may also be water soluble. An ointment may also comprise from about 2% to about 10% of an emollient plus from about 0.1% to about 2% of a thickening agent. A more complete disclosure of thickening agents useful herein can be found in Segarin, Cosmetics, Science and Technology, 2nd Edition, Vol. 1, pp. 72-73 (1972).

If the carrier is formulated as an emulsion, from about 1% to about 10%, preferably from about 2% to about 5%, of the carrier system comprises an emulsifier. Emulsifiers may be nonionic, anionic or cationic. Suitable emulsifiers are disclosed in, for example, U.S. Pat. No. 3,755,560, issued Aug. 28, 1973, Dickert et al; U.S. Pat. No. 4,421,769, issued Dec. 20, 1983, Dixon et al.; and McCutcheon's Detergents and Emulsifiers, North American Edition, pages 317-324 (1986); the disclosures of which are incorporated herein by reference. Preferred emulsifiers are anionic or nonionic, although the other types may also be used.

Lotions and creams can be formulated as emulsions as well as solutions. Preferably such lotions comprise from about 0.1% to about 20%, more preferably from about 1% to about 5%, of the active compound; from about 1% to about 20%, preferably from about 5% to about 10%, of an emollient; from about 25% to about 75%, preferably from about 45% to about 95%, water; and from about 0.1% to about 10%, preferably from about 0.5% to about 5%, of an emulsifier. Such creams would preferably comprise from about 0.1% to about 20%, more preferably from about 1% to about 5%, of the active compound; from about 1% to about 20%, preferably from about 5% to about 10%, of an emollient; from about 20% to about 80%, preferably from about 30% to about 70%, water; and from about 1% to about 10%, preferably from about 2% to about 5%, of an emulsifier.

Single emulsion skin care preparations, such as lotions and creams, of the oil-in-water type and water-in-oil type are well-known in the cosmetic art and are useful in the present invention. Multiphase emulsion compositions, such as the water-in-oil-in-water type, as disclosed in U.S. Pat. No. 4,254,105, Fakuda et al., issued Mar. 3, 1981, incorporated herein by reference, are also useful in the present invention. In general, such single or multiphase emulsions contain water, emollients and emulsifiers as essential ingredients.

Triple emulsion carrier systems comprising an oil-in-water-in-silicone fluid emulsion composition as disclosed in U.S. Pat. No. 4,960,764, Figueroa, issued Oct. 2, 1990, are also useful in the present invention. Preferably, this triple emulsion carrier system can be combined with from about 0.1% to about 20%, more preferably from about 1% to about 5%, of the active compound to yield the topical pharmaceutical composition of the present invention.

Another emulsion carrier system useful in the topical pharmaceutical compositions of the present invention is a micro-emulsion carrier system. Such a system comprises from about 9% to about 15% squalane; from about 25% to about 40% silicone oil; from about 8% to about 20% of a fatty alcohol; from about 15% to about 30% of polyoxyethylene sorbitan mono-fatty acid (commercially available under the trade name Tweens) or other nonionics; and from about 7% to about 20% water. This carrier system is preferably combined with from about 1% to about 5% of the active compound.

If the topical pharmaceutical compositions of the present invention are formulated as a gel or a cosmetic stick, a suitable amount of a thickening agent, as disclosed supra, is added to a cream or lotion formulation.

Various water-soluble materials may also be present in the compositions of this invention. These include humectants, proteins and polypeptides, preservatives and an alkaline agent. In addition, the topical compositions herein can contain conventional cosmetic adjuvants, such as dyes, opacifiers (e.g., titanium dioxide), pigments and perfumes.

Formulations of the invention suitable for oral administration may be in the form of capsules, cachets, pills, wafers, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention as an active ingredient. A compound of the invention may also be administered as a bolus, electuary or paste.

In solid dosage forms of the invention for oral administration (capsules, tablets, wafers, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of a creatine compound include pharmaceutically acceptable emulsions, microemulsions, solutions, suspensions, syrups and elixirs. In addition to the active ingredient, the liquid dosage forms may contain inert dilutents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert dilutents, oral or opthalmic compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

### EQUIVALENTS

Those skilled in the art will recognize, or be able to ascertain using no more than routine experimentation, many equivalents to the specific embodiments of the invention described herein. Such equivalents are intended to be encompassed by the following claims.

The entire contents of all references, patents, and patent applications cited herein are expressly incorporated by reference.

1. A method for treating dermatitis in a subject, comprising administering to said subject an effective amount of a creatine compound, such that said dermatitis is treated.

2. The method of claim 1, wherein said subject is a mammal.

3. The method of claim 2, wherein said subject is a human.

4. The method of any one of claims 1-3, wherein said creatine compound is administered in combination with a pharmaceutically acceptable carrier.

5. The method of claim 4, wherein said pharmaceutically acceptable carrier is suitable for topical or oral administration.

6. The method of any one of claims 1-5, further comprising administering the creatine compound in combination with an anti-inflammatory agent.

7. The method of any one of claims 1-6, wherein said dermatitis is atopic dermatitis, contact dermatitis, generalized exfoliative dermatitis, neurodermatitis, nummular dermatitis, seborrheic dermatitis, stasis dermatitis, perioral dermatitis or pompholyx.

8. The method of any one of claims 1-7, wherein said creatine compound is creatine, creatine citrate, creatine ascorbate, creatine pyruvate, creatine monohydrate, cyclocreatine, or creatine phosphate.

9. A method for treating a subject for dermatitis, comprising administering an effective amount of a creatine compound to a subject such that the subject is treated, wherein the creatine compound is of the general formula: 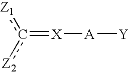
and pharmaceutically acceptable salts thereof, where:
a) Y is selected from the group consisting of: —CO₂H, —NHOH, —NO₂, —SO₃H, —C(═O)NHSO₂J and —P(═O)(OH)(OJ), wherein J is selected from the group consisting of: hydrogen, C₁-C₆ straight chain alkyl, C₃-C₆ branched alkyl, C₂-C₆ alkenyl, C₃-C₆ branched alkenyl, and aryl;
b) A is selected from the group consisting of: C, CH, C₁-C₅ alkyl, C₂-C₅ alkenyl, C₂-C₅ alkynyl, and C₁-C₅ alkoyl chain, each having 0-2 substituents which are selected independently from the group consisting of:
1) K, where K is selected from the group consisting of: C₁-C₆ straight alkyl, C₂-C₆ straight alkenyl, C₁-C₆ straight alkoyl, C₃-C₆ branched alkyl, C₃-C₆ branched alkenyl, and C₄-C₆ branched alkoyl, K having 0-2 substituents independently selected from the group consisting of: bromo, chloro, epoxy and acetoxy;
2) an aryl group selected from the group consisting of: a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy; and
3) —NH-M, wherein M is selected from the group consisting of: hydrogen, C₁-C₄ alkyl, C₂-C₄ alkenyl, C₁-C₄ alkoyl, C₃-C₄ branched alkyl, C₃-C₄ branched alkenyl, and C₄ branched alkoyl;
c) X is selected from the group consisting of NR₁, CHR₁, CR₁, O and S, wherein R₁ is selected from the group consisting of:
1) hydrogen;
2) K where K is selected from the group consisting of: C₁-C₆ straight alkyl, C₂-C₆ straight alkenyl, C₁-C₆ straight alkoyl, C₃-C₆ branched alkyl, C₃-C₆ branched alkenyl, and C₄-C₆ branched alkoyl, K having O-2 substituents independently selected from the group consisting of: bromo, chloro, epoxy and acetoxy;
3) an aryl group selected from the group consisting of a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy;
4) a C₅-C₉ α-amino-ω-methyl-ω-adenosylcarboxylic acid attached via the ω-methyl carbon;
5) a C₅-C₉ α-amino-ω-aza-ω-methyl-w-adenosylcarboxylic acid attached via the ω-methyl carbon; and
6) a C₅-C₉ α-amino-ω-thia-ωmethyl-w-adenosylcarboxylic acid attached via the w-methyl carbon;
d) Z₁ and Z₂ are chosen independently from the group consisting of: ═O, —NHR₂, —CH₂R₂, —NR₂OH; wherein Z₁ and Z₂ may not both be ═O and wherein R₂ is selected from the group consisting of:
1) hydrogen;
2) K, where K is selected from the group consisting of: C₁-C₆ straight alkyl; C₂-C₆ straight alkenyl, C₁-C₆ straight alkoyl, C₃-C₆ branched alkyl, C₃-C₆ branched alkenyl, and C₄-C₆ branched alkoyl, K having O-2 substituents independently selected from the group consisting of: bromo, chloro, epoxy and acetoxy;
3) an aryl group selected from the group consisting of a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy;
4) a C₄-C₈ α-amino-carboxylic acid attached via the ω-carbon;
5) B, wherein B is selected from the group consisting of: —CO₂H, —NHOH, —SO₃H, —NO₂, OP(═O)(OH)(OJ) and —P(═O)(OH)(OJ), wherein J is selected from the group consisting of: hydrogen, C₁-C₆ straight alkyl, C₃-C₆ branched alkyl, C₂-C₆ alkenyl, C₃-C₆ branched alkenyl, and aryl, wherein B is optionally connected to the nitrogen via a linker selected from the group consisting of: C₁-C₂ alkyl, C₂ alkenyl, and C₁-C₂ alkoyl;
6)-D-E, wherein D is selected from the group consisting of: C₁-C₃ straight alkyl, C₃ branched alkyl, C₂-C₃ straight alkenyl, C₃ branched alkenyl, C₁-C₃ straight alkoyl, aryl and aroyl; and E is selected from the group consisting of: —(PO₃)ₙ NMP, where n is 0-2 and NMP is ribonucleotide monophosphate connected via the 5′-phosphate, 3′-phosphate or the aromatic ring of the base; —[P(═O)(OCH₃)(O)]ₘ-Q, where m is 0-3 and Q is a ribonucleoside connected via the ribose or the aromatic ring of the base; —[P(═O)(OH)(CH₂)]ₘ-Q, where m is 0-3 and Q is a ribonucleoside connected via the ribose or the aromatic ring of the base; and an aryl group containing 0-3 substituents chosen independently from the group consisting of: Cl, Br, epoxy, acetoxy, —OG, —C(═O)G, and —CO₂G, where G is independently selected from the group consisting of: C₁-C₆ straight alkyl, C₂-C₆ straight alkenyl, C₁-C₆ straight alkoyl, C₃-C₆ branched alkyl, C₃-C₆ branched alkenyl, C₄-C₆ branched alkoyl, wherein E may be attached to any point to D, and if D is alkyl or alkenyl, D may be connected at either or both ends by an amide linkage; and
7) -E, wherein E is selected from the group consisting of —(PO₃)ₙNMP, where n is 0-2 and NMP is a ribonucleotide monophosphate connected via the 5′-phosphate, 3′-phosphate or the aromatic ring of the base; —[P(═O)(OCH₃)(O)]ₘ-Q, where m is 0-3 and Q is a ribonucleoside connected via the ribose or the aromatic ring of the base; —[P(═O)(OH)(CH₂)]ₘ-Q, where m is 0-3 and Q is a ribonucleoside connected via the ribose or the aromatic ring of the base; and an aryl group containing 0-3 substituents chose independently from the group consisting of: Cl, Br, epoxy, acetoxy, —OG, —C(═O)G, and —CO₂G, where G is independently selected from the group consisting of: C₁-C₆ straight alkyl, C₂-C₆ straight alkenyl, C₁-C₆ straight alkoyl, C₃-C₆ branched alkyl, C₃-C₆ branched alkenyl, C₄-C₆ branched alkoyl; and if E is aryl, E may be connected by an amide linkage;
e) if R₁ and at least one R₂ group are present, R₁ may be connected by a single or double bond to an R₂ group to form a cycle of 5 to 7 members;
f) if two R₂ groups are present, they may be connected by a single or a double bond to form a cycle of 4 to 7 members; and
g) if R₁ is present and Z₁ or Z₂ is selected from the group consisting of —NHR₂, —CH₂R₂ and —NR₂OH, then R₁ may be connected by a single or double bond to the carbon or nitrogen of either Z₁ or Z₂ to form a cycle of 4 to 7 members.

10. The method of claim 9, wherein said creatine compound is creatine, creatine phosphate, cyclocreatine, cyclocreatine phosphate, creatine monohydrate, creatine pyruvate, creatine ascorbate, homocyclocreatine, 3-guanidinopropionic acid, guanidinoacetate, or a guanidino benzoic acid.

11. The method of claim 9 or claim 10, further comprising co-administering to said subject an effective amount of an anti-inflammatory agent.

12. The method of any one of claims 9-11, further comprising administering a pharmaceutical carrier suitable for topical or oral administration.

13. The method of claim 12, wherein said creatine compound is administered in a lotion, cream, mousse, aerosol, gel, emulsion, solution, ointment or solid.

14. A method for treating a subject for dermatitis comprising modulation of energy of skin by administering an effective amount of a creatine compound such that the subject is treated.

15. The method of claim 14, wherein said modulation occurs by modulating creatine kinase.

16. A composition for treating dermatitis in a subject, comprising an effective amount of a creatine compound or a salt thereof, and a pharmaceutically acceptable carrier.

17. The composition of claim 16, wherein said composition is suitable for topical or oral administration.

18. The composition of claim 17, wherein said composition is a lotion, cream, mousse, aerosol, gel, emulsion, solution, ointment or solid.

19. The composition of any one of claims 16-18, wherein said effective amount is effective to treat dermatitis.

20. The composition of any one of claims 16-18, wherein said effective amount is effective to prevent dermatitis.

21. The composition of any one of claims 16-20, wherein said creatine compound is creatine creatine phosphate, cyclocreatine, cyclocreatine phosphate, creatine monohydrate, creatine pyruvate, creatine ascorbate, homocyclocreatine, 3-guanidinopropionic acid, guanidinoacetate, or a guanidino benzoic acid.

22. A composition for treating dermatitis comprising an effective amount of a creatine compound and a pharmaceutical carrier suitable for topical administration, wherein said creatine compound is of the general formula: 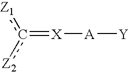
and pharmaceutically acceptable salts thereof, where:
a) Y is selected from the group consisting of: —CO₂H, —NHOH, —NO₂, —
SO₃H, —C(═O)NHSO₂J and —P(═O)(OH)(OJ), wherein J is selected from the group consisting of: hydrogen, C₁-C₆ straight chain alkyl, C₃-C₆ branched alkyl, C₂-C₆ alkenyl, C₃-C₆ branched alkenyl, and aryl;
b) A is selected from the group consisting of: C, CH, C₁-C₅ alkyl, C₂-C₅ alkenyl, C₂-C₅ alkynyl, and C₁-C₅ alkoyl chain, each having 0-2 substituents which are selected independently from the group consisting of:
1) K, where K is selected from the group consisting of: C₁-C₆ straight alkyl, C₂-C₆ straight alkenyl, C₁-C₆ straight alkoyl, C₃-C₆ branched alkyl, C₃-C₆ branched alkenyl, and C₄-C₆ branched alkoyl, K having 0-2 substituents independently selected from the group consisting of: bromo, chloro, epoxy and acetoxy;
2) an aryl group selected from the group consisting of: a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy; and
3)-NH-M, wherein M is selected from the group consisting of: hydrogen, C₁-C₄ alkyl, C₂-C₄ alkenyl, C₁-C₄ alkoyl, C₃-C₄ branched alkyl, C₃-C₄ branched alkenyl, and C₄ branched alkoyl;
c) X is selected from the group consisting of NR₁, CHR₁, CR₁, O and S, wherein R₁ is selected from the group consisting of:
1) hydrogen;
2) K where K is selected from the group consisting of: C₁-C₆ straight alkyl, C₂-C₆ straight alkenyl, C₁-C₆ straight alkoyl, C₃-C₆ branched alkyl, C₃-C₆ branched alkenyl, and C₄-C₆ branched alkoyl, K having O-2 substituents independently selected from the group consisting of: bromo, chloro, epoxy and acetoxy;
3) an aryl group selected from the group consisting of a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy;
4) a C₅-C₉ α-amino-ω-methyl-ω-adenosylcarboxylic acid attached via the ω-methyl carbon;
5) a C₅-C₉ α-amino-ω-aza-ω-methyl-w-adenosylcarboxylic acid attached via the ω-methyl carbon; and
6) a C₅-C₉ α-amino-ω-thia-ωmethyl-w-adenosylcarboxylic acid attached via the w-methyl carbon;
d) Z₁ and Z₂ are chosen independently from the group consisting of: ═O, —NHR₂, —CH₂R₂, —NR₂OH; wherein Z₁ and Z₂ may not both be ═O and wherein R₂ is selected from the group consisting of:
1) hydrogen;
2) K, where K is selected from the group consisting of: C₁-C₆ straight alkyl; C₂-C₆ straight alkenyl, C₁-C₆ straight alkoyl, C₃-C₆ branched alkyl, C₃-C₆ branched alkenyl, and C₄-C₆ branched alkoyl, K having O-2 substituents independently selected from the group consisting of: bromo, chloro, epoxy and acetoxy;
3) an aryl group selected from the group consisting of a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy;
4) a C₄-C₈ α-amino-carboxylic acid attached via the ω-carbon;
5) B, wherein B is selected from the group consisting of: —CO₂H, —NHOH, —SO₃H, —NO₂, OP(═O)(OH)(OJ) and —P(═O)(OH)(OJ), wherein J is selected from the group consisting of: hydrogen, C₁-C₆ straight alkyl, C₃-C₆ branched alkyl, C₂-C₆ alkenyl, C₃-C₆ branched alkenyl, and aryl, wherein B is optionally connected to the nitrogen via a linker selected from the group consisting of: C₁-C₂ alkyl, C₂ alkenyl, and C₁-C₂ alkoyl;
6)-D-E, wherein D is selected from the group consisting of: C₁-C₃ straight alkyl, C₃ branched alkyl, C₂-C₃ straight alkenyl, C₃ branched alkenyl, C₁-C₃ straight alkoyl, aryl and aroyl; and E is selected from the group consisting of: —(PO₃)ₙ NMP, where n is 0-2 and NMP is ribonucleotide monophosphate connected via the 5′-phosphate, 3′-phosphate or the aromatic ring of the base; —[P(═O)(OCH₃)(O)]ₘ-Q, where m is 0-3 and Q is a ribonucleoside connected via the ribose or the aromatic ring of the base; —[P(═O)(OH)(CH₂)]ₘ-Q, where m is 0-3 and Q is a ribonucleoside connected via the ribose or the aromatic ring of the base; and an aryl group containing 0-3 substituents chosen independently from the group consisting of: Cl, Br, epoxy, acetoxy, —OG, —C(═O)G, and —CO₂G, where G is independently selected from the group consisting of: C₁-C₆ straight alkyl, C₂-C₆ straight alkenyl, C₁-C₆ straight alkoyl, C₃-C₆ branched alkyl, C₃-C₆ branched alkenyl, C₄-C₆ branched alkoyl, wherein E may be attached to any point to D, and if D is alkyl or alkenyl, D may be connected at either or both ends by an amide linkage; and
7) -E, wherein E is selected from the group consisting of —(PO₃)ₙNMP, where n is 0-2 and NMP is a ribonucleotide monophosphate connected via the 5′-phosphate, 3′-phosphate or the aromatic ring of the base; —[P(═O)(OCH₃)(O)]ₘ-Q, where m is 0-3 and Q is a ribonucleoside connected via the ribose or the aromatic ring of the base; —[P(═O)(OH)(CH₂)]ₘ-Q, where m is 0-3 and Q is a ribonucleoside connected via the ribose or the aromatic ring of the base; and an aryl group containing 0-3 substituents chose independently from the group consisting of: Cl, Br, epoxy, acetoxy, —OG, —C(═O)G, and —CO₂G, where G is independently selected from the group consisting of: C₁-C₆ straight alkyl, C₂-C₆ straight alkenyl, C₁-C₆ straight alkoyl, C₃-C₆ branched alkyl, C₃-C₆ branched alkenyl, C₄-C₆ branched alkoyl; and if E is aryl, E may be connected by an amide linkage;
e) if R₁ and at least one R₂ group are present, R₁ may be connected by a single or double bond to an R₂ group to form a cycle of 5 to 7 members;
f) if two R₂ groups are present, they may be connected by a single or a double bond to form a cycle of 4 to 7 members; and
g) if R₁ is present and Z₁ or Z₂ is selected from the group consisting of —NHR₂, —CH₂R₂ and —NR₂OH, then R₁ may be connected by a single or double bond to the carbon or nitrogen of either Z₁ or Z₂ to form a cycle of 4 to 7 members.

23. The composition of claim 22, further comprising an anti-inflammatory agent.

24. A packaged pharmaceutical composition, comprising an effective amount of a creatine compound in combination with a pharmaceutically acceptable carrier, and instructions for using the compound to treat dermatitis.

25. The pharmaceutical composition of claim 24, further comprising an anti-inflammatory agent.
